(19) United States
(12) Patent Application Publication
(10) Pub. No.: US 2023/0098743 A1
(43) Pub. Date: Mar. 30, 2023

(54) BACTERIAL STRAINS AND COMPOSITIONS THEREOF FOR ORAL USE IN THE TREATMENT OF VIRAL INFECTIONS OF THE RESPIRATORY SYSTEM

(71) Applicant: SOFAR S.P.A., Trezzano Rosa (MI) (IT)

(72) Inventors: Andrea BIFFI, Trezzano Rosa (MI) (IT); Walter FIORE, Trezzano Rosa (MI) (IT)

(21) Appl. No.: 17/798,056

(22) PCT Filed: Mar. 9, 2021
(86) PCT No.: PCT/IB2021/051954

§ 371 Date: Aug. 5, 2022

(30) Foreign Application Priority Data
Mar. 9, 2020 (IT) 102020000005011
Mar. 24, 2020 (IT) 102020000006205

(51) Int. Cl. A61K 35/747; A61K 35/745; A61P 31/14

(52) U.S. Cl. CPC A61K 35/747; A61K 35/745; A61P 31/14

(57) ABSTRACT

The present invention relates to bacterial strains and compositions thereof for use in the treatment of viral infections of the respiratory tract, preferably from coronavirus, such as for example a severe acute respiratory syndrome coronavirus (COVID-19), by stimulating and/or modulating cytokines and/or markers involved in the inflammatory processes of said viral infections of the respiratory tract.

The present invention relates to specific and selected bacterial strains and compositions thereof for use in the treatment of viral infections of the respiratory tract, preferably from coronavirus, such as for example a severe acute respiratory syndrome coronavirus (COVID-19), by stimulating and/or modulating cytokines and/or markers involved in the inflammatory processes of said viral infections of the respiratory tract. In particular, said specific bacterial strains and the compositions thereof are for use in the stimulation and/or modulation of cytokine profiles and/or inflammatory/immune pathways for reducing the expression of at least one pro-inflammatory marker and/or stimulating the expression of at least one anti-inflammatory marker.

Viral infections of the respiratory tract, as the name says, are infectious diseases caused by viruses that affect the organs of the upper and/or lower respiratory system (nose, pharynx, larynx, trachea, bronchi and lungs). In particular, the present invention relates to viral infections of the respiratory tract preferably caused by at least one coronavirus, such as for example a severe acute respiratory syndrome, abbreviated as SARS.

In the context of the present invention, the term “coronavirus” is used to indicate a virus of the Coronaviridae family, subfamily: Coronavirinae, genus: Betacoronavirus, species: severe acute respiratory syndrome-related coronavirus (in short, SARSr-CoV or SARS-coronavirus or coronavirus), preferably selected from the following strains: (I) severe acute respiratory syndrome coronavirus (SARS-CoV) (isolated and identified for the first time in 2002), (II) severe acute respiratory syndrome coronavirus-2 (SARS-CoV-2) (isolated and identified for the first time in 2019), and (III) severe acute respiratory syndrome coronavirus-like (SARS-CoV-like).

The coronavirus of the (I) SARS-CoV strain was the origin of the SARS epidemic in 2002-2003, which infected some 8,000 people in Asia, causing about 700 deaths. The coronavirus of the (II) SARS-CoV-2 strain was the origin of the 2019-2020 SARS epidemic that began in China and spread around the world; the SARS epidemic caused by said SARS-CoV-2 strain is referred to as COVID-19 (COronaVIrus Disease 19, also known as severe acute respiratory syndrome coronavirus 2 - SARS-CoV-2 - or coronavirus disease 2019 or coronavirus syndrome 2019).

It is thought that there are some factors that can affect the composition of an individual’s gut and/or lung microbiota, such as for example contact with pathogens, diet and lifestyle, drug intake and/or bacterial strains.

Following an intense and extensive research and development activity, the Applicant identified, isolated and studied specific bacterial strains (for example, probiotic bacterial strains or derivatives thereof) and compositions thereof capable of significantly stimulating and/or modulating suitable cytokine profiles and/or inflammatory/immune pathways, such as for example interleukin IL-6, IL-8, IL-15, IL-1a, IL-1β, IL-2, IL-2 (IL-2R) receptor, iNOS, NO, TLR-4 protein, TNF-alpha and/or interleukin IL-10, so as to treat viral infections of the respiratory tract (upper and lower respiratory tract), in particular viral infections of the respiratory tract caused by at least one virus of the species coronavirus (for example SARS-CoV-2 or virus responsible for COVID 19 disease), maintaining the gut microbiota balance and the boosting thereof, while at the same time limiting the intestinal translocation of bacterial pathogens and, thus, the risk of secondary bacterial infections.

The bacterial strains isolated and selected in the present invention are capable of modulating some inflammatory/immune pathways reducing the expression levels of one or more pro-inflammatory markers and/or stimulating the expression levels of one or more anti-inflammatory markers (for example cytokines and other biomarkers). For this reason, said specific bacterial strains and the compositions thereof are validly used in the stimulation and/or modulation of cytokines and/or markers involved in inflammatory processes for the treatment of viral infections of the respiratory tract; advantageously, said bacterial strains and the compositions thereof are validly used in a method for the treatment of viral infections of the respiratory system from coronavirus, preferably of a severe acute respiratory syndrome coronavirus (e.g. COVID-19).

In detail, for the purposes of the present invention, the Applicant identified, isolated and studied bacterial strains belonging to the genus Lactobacillus or Bifidobacterium; preferably bacterial strain belonging to a species selected from: Lactobacillus paracasei, Lactobacillus plantarum, Bifidobacterium breve, Bifidobacterium animalis subsp. lactis, Bifidobacterium bifidum, more preferably bacterial strains (in short, bacterial strains or bacterial strains of the invention) identified as: Lactobacillus paracasei DG® (trademark registered by SOFAR S.p.A.) deposited under the accession number CNCM I-1572 (in short, DG®), Lactobacillus paracasei LPC-S01 deposited under the accession number DSM 26760 (in short, LPC-S01), Bifidobacterium breve BbIBS01 deposited under the accession number DSM 33231 (in short, BbIBS01), Bifidobacterium breve BbIBS02 deposited under the accession number DSM 33232 (in short, BbIBS02), Bifidobacterium animalis subsp. lactis BIIBS01 deposited under the accession number DSM 33233 (in short, BIIBS01), Lactobacillus plantarum LpIBS01 deposited under the accession number DSM 33234 (in short, LpIBS01), and Bifidobacterium bifidum MIMBb23sg or BbfIBS01 deposited under the accession number DSM 32708 (in short, MIMBb23sg or BbfIBS01).

All the bacterial strains mentioned in the present invention were deposited according to the provisions pursuant to the Budapest treaty. The Depositing party of the bacterial strains described and/or claimed in the present patent application and the proprietor thereof express, from the outset, their consent to make available all the above strains for the whole duration of the patent.

The bacterial strains of the present invention and the compositions thereof preferably for oral use, as specified in the present invention, are effective as modulators of inflammatory/immune pathways in the significant reduction of the expression levels of at least one pro-inflammatory marker, such as for example IL-6, IL-8, IL-15, IL-1a, IL-1β, IL-2, IL-2 (IL-2R) receptor, iNOS, NO and/or TLR-4 protein, and/or in the significant stimulation (or increase) of the expression levels of at least one anti-inflammatory marker, such as for example IL-10.

Consequently, the bacterial strains of the present invention and the compositions thereof, by virtue of their modulating effect on the expression levels of the aforementioned cytokines and/or biomarkers, are effective in the treatment of viral infections and/or inflammations of the respiratory tract, preferably from coronavirus, such as for example, severe acute respiratory syndrome (SARS or COVID-19), and symptoms or disorders related thereto.

In particular, the bacterial strains belonging to the species Lactobacillus paracasei, preferably Lactobacillus paracasei DG® (CNCM I-1572), have demonstrated an antiviral activity against SARS-CoV-2, Various modes of antiviral action have been proposed for probiotic bacterial strains, including: direct interaction between bacterial strains and viruses, production of antiviral substances and stimulation of the host’s immune system. In the context of SARS-CoV-2 infection, probiotic bacterial strains, preferably belonging to the genus Lactobacillus, can act as a barrier against the penetration of the virus into the host cells through various mechanisms. Furthermore, the administration of probiotic bacterial strains before, during or after COVID-19 infection increases the natural immunity of the subject.

The results reported in the present description show both the activity of boosting the antiviral immune system by the bacterial strains of the present invention, preferably belonging to the species Lactobacillus paracasei (for example Lactobacillus paracasei DG® CNCM I-1572), and their ability to prevent the replication of SARS-CoV-2 through in vitro experiments.

Among the probiotic strains tested, the bacterial strains belonging to the species Lactobacillus paracasei, preferably Lactobacillus paracasei DG® (CNCM I-1572), proved to be the most promising in terms of antiviral immunomodulatory activity, capable of inducing the expression of IFN and genes involved in the antiviral response signalling pathways such as TLR7, IFIH, IRF3, IRF7 and MAVS.

This is of particular interest in the context of SARS-CoV-2 infection. Coronaviruses have various mechanisms to circumvent the innate immune response, especially by modifying the Type I IFN response. Compared to other respiratory viruses, SARS-CoV-2 induces a lower antiviral transcriptional response, characterised by low levels of type I IFN and high chemokine expression. Furthermore, patients with severe COVID-19 have shown a reduced type I IFN response and a lower viral clearance. Furthermore, TLR7 has been implicated as an important pattern recognition receptor in the recognition of ssRNA of Middle Eastern Respiratory Syndrome CoV (MERS-CoV) and severe acute respiratory syndrome CoV (SARS-CoV) in murine infection models, making it a likely candidate to function as a central pattern recognition receptor in SARS-CoV-2. Sequencing of the entire genome of SARS-CoV, MERS-CoV and SARS-CoV-2 has shown that the SARS-COV-2 genome contains more ssRNA patterns that could interact with TLR7 than the SARS-CoV genome, indicating that TLR7 signalling could be even more relevant in the pathogenesis of COVID-19. Rare putative variants with loss of TLR7 X chromosome function - which were associated with altered type I and II IFN responses - were identified in several cases of young male patients with severe COVID-19.

An unbalanced immune response, characterised by a weak production of type I interferons (IFN-Is) and an exacerbated release of proinflammatory cytokines contributes to the severe forms of COVID-19. Furthermore, chronic low-grade systemic inflammation accompanies various comorbidities that adversely affect the outcomes of patients with COVID-19.

The results reported in the present description show that in vitro prophylactic treatment or simultaneous co-treatment with bacterial strains belonging to the species Lactobacillus paracasei, preferably Lactobacillus paracasei DG® (CNCM I-1572), suppressed the inflammatory response triggered by the SARS-CoV-2 infection in Caco-2 cells, given that the transcription levels of the IL-6, IL8 and TSLP1 proinflammatory cytokines were reduce with respect to the control.

Furthermore, it was also observed that the combination of the bacterial strains Lactobacillus paracasei DG® (CNCM I-1572) and Lactobacillus paracasei LPC-S01 (DSM 26760) positively modulated the antiviral immune responses to a greater extent with respect to the strain Lactobacillus rhamnosus GG (ATCC 53103), further showing an action in decreasing viral replication and in modulating proinflammatory responses induced by the SARS-CoV-2 virus, even in this case to a greater extent with respect to the strain Lactobacillus rhamnosus GG (ATCC 53103).

Thus, the preventive and/or curative use of bacterial strains according to the present invention, preferably belonging to the species Lactobacillus paracasei (for example Lactobacillus paracasei DG® CNCM I-1572 or the combination of Lactobacillus paracasei DG® (CNCM I-1572) and Lactobacillus paracasei LPC-S01 (DSM 26760), contributes towards alleviating the excessive inflammatory response induced by SARS-CoV-2 infection.

The bacterial strains of the present invention and the compositions thereof have no relevant side effects and they can be administered to all categories of subjects in need, including the elderly, pregnant or breastfeeding women, paediatric subjects (0-12 years), subjects with cardiovascular complications, subjects with diabetes, immunocompromised subjects (due to congenital or acquired disease or under treated with immunosuppressants or subjects who have undergone transplant surgery) or subjects with other comorbidities.

Furthermore, the bacterial strains of the present invention and the compositions thereof are easy to prepare and cost-effective.

These and other objects, which will be clear from the detailed description that follows, are attained by the bacterial strains, by the compositions and by the mixtures of the present invention due to the technical characteristics reported in the description and claimed in the attached claims.

### DESCRIPTION OF THE FIGURES

FIGS. 1°-C schematically represent the drawings of in vitro studies of evaluation of antiviral responses in Caco-2 intestinal epithelial cells following: (A) absence of treatment with a bacterial strain according to the present invention, (B) a pre-treatment with a bacterial strain according to the present invention, and (C) a co-treatment with a bacterial strain according to the present invention, each with respect to a treatment with SARS-CoV-2 virus.

FIGS. 2A-C represent the effect of a bacterial strain according to the present invention on a panel of cytokines/chemokines and molecules having antiviral action or involved in the antiviral responses produced by the Caco-2 intestinal epithelial cells, compared with the effect of the strain L. rhamnosus GG ATCC 53103.

FIGS. 3A-C represent the effect of a bacterial strain according to the present invention on a panel of cytokines/chemokines and molecules with antiviral action or involved in antiviral responses produced by Caco-2 intestinal epithelial cells following a pre-treatment with said composition with respect to a treatment with SARS-CoV-2 virus.

FIGS. 4A-C represent the expression level of virus-specific genes that encode RNA-dependent RNA polymerase (RdRp) and gene E (CoVE), and the cytokine expression profile (pro-inflammatory and anti-inflammatory) of SARS-CoV-2 in vitro infected Caco-2 cells pre-treated or not-treated with a probiotic bacterial strain according to the present invention, and compared with the effect of the strain L. rhamnosus GG ATCC 53103.

FIGS. 5A-B represent the expression level of virus-specific genes that encode RNA-dependent RNA polymerase (RdRp) and gene E (CoVE), and the inflammatory cytokine expression profile of SARS-CoV-2 in vitro infected Caco-2 cells co-treated or not treated with a probiotic bacterial strain according to the present invention, and compared with the effect of the strain L. rhamnosus GG ATCC 53103.

### DETAILED DESCRIPTION OF THE INVENTION

It is believed that the bacterial strains of the present invention can play a role in the involvement of the gastrointestinal tract in viral infections of the respiratory tract. As a matter of fact, the gut microbiota is closely linked to the lung microbiota through the gut-lung axis. Within the gut-lung axis, bacteria, viruses and mycetes interact closely with each other through direct and indirect mechanisms, particularly involving immune/inflammatory cells. An alteration of the gut microbiota could result in an increase in intestinal permeability, with an increased risk of translocation of pathogens (e.g. bacteria and/or viruses), which could have a negative impact on the lung microbiota.

In literature, there has been reported the presence of viral nucleic acids in faecal samples of some patients with COVID-19 and it has been reported that the virus binds to the ACE2 receptor, which is found in the lungs and small intestine epithelial cells.

Based on gut -lung axis cross-talk, it has been assumed that, although there is no direct clinical evidence that the modulation of gut microbiota plays a therapeutic role in the treatment of COVID-19, probiotic bacterial strains can modulate the gut microbiota to favourably affect gut symptoms and protect the respiratory system.

Furthermore, in infections of the respiratory tract from coronavirus (for example, COVID-19), there has been observed an increase in some pro-inflammatory markers, such as IL-6, IL-8, IL-1 the IL-1 family (particularly IL-1b), and IL-2R (IL is the abbreviation of interleukin), and a decrease in anti-inflammatory markers, such as IL-10, probably due to an alteration of the gut microbiota characterised by a reduction of bacteria belonging to the genera Lactobacillus and Bifidobacterium.

For example, it has been observed that when the SARS-CoV-2 virus infects the upper and lower respiratory tract may cause mild or very acute respiratory syndrome resulting in the release of pro-inflammatory cytokines, including IL-1β and IL-6.

Furthermore, it has been observed that there are statistically significant differences (p <0.05) in the expression levels of interleukin-2 receptor (IL-2R) and of IL-6 in the serum of patients with coronavirus infection with mild or serious or critical disease severity (p <0.05); the increased expression of IL-2R and IL-6 in serum may predict the severity of 2019-nCoV pneumonia and prognosis of the patients.

Thus, suppression of pro-inflammatory cytokines of the IL-1 family and especially of the IL-6 interleukin has been shown to have a therapeutic effect in many inflammatory diseases, including viral infections of the respiratory tract, preferably from coronavirus.

Bacterial strains, such as probiotics or derivatives thereof, are capable of benefitting the gut microbiota balance and thus restoring the altered intestinal barrier and hindering the translocation of bacterial or viral pathogens, through the production of SCFAs (short-chain fact acids), the increase in the expression of proteins which form tight-junctions such as occludin and zonulin, thus improving the barrier function, and a positive modulation of the various inflammatory/immune pathways.

Forming an object of the present invention is a bacterial strain for use in a method for the preventive and/or curative treatment of viral infections and/or inflammations of the respiratory system in a subject in need, preferably viral infections of the respiratory system from a coronavirus, more preferably a severe acute respiratory syndrome coronavirus (e.g. SARS-CoV or COVID-19), and of diseases or symptoms or disorders related thereto or deriving therefrom, wherein said bacterial strain belongs to the genus Lactobacillus or Bifidobacterium, preferably wherein said bacterial strain belongs to a species selected from: Lactobacillus paracasei, Lactobacillus plantarum, Bifidobacterium breve, Bifidobacterium animalis subsp. lactis and Bifidobacterium bifidum, even more preferably wherein said bacterial strain belongs to the species Lactobacillus paracasei, such as for example Lactobacillus paracasei DG® (CNCM I-1572) and/or Lactobacillus paracasei LPC-S01 (DSM 26760).

Forming an object of the present invention is at least one bacterial strain (in short, bacterial strain/s of the invention) selected from the group comprising or, alternatively, consisting of:
(a) a bacterial strain belonging to the species Lactobacillus paracasei identified as Lactobacillus paracasei DG® (trademark registered by SOFAR S.p.A.) and deposited at the National Collection of Cultures of Microorganisms of the Pasteur Institute in Paris under the accession number CNCM I-1572 (deposited on 5 May 1995 by Sofar S.p.A as Lactobacillus casei ssp. casei under N° CNCM I-1572 and subsequently reclassified as Lactobacillus paracasei CNCM I-1572; it should be observed that it is still and exclusively the same bacterial strain irrespective of the name Lactobacillus casei DG® CNCM I-1572 or Lactobacillus paracasei DG® CNCM I-1572),
(b) a bacterial strain belonging to the species Lactobacillus paracasei identified as Lactobacillus paracasei LPC-S01 and deposited at Deutsche Sammlung von Mikroorganismen und Zellkulturen GmbH (DSMZ) under the accession number DSM 26760 (deposited on 11 Jan. 2013 by Sofar S.p.A and on 15 May 2017 requested the conversion of the deposit into a deposit according to the Budapest Treaty),
(c) a bacterial strain belonging to the species Bifidobacterium breve identified as Bifidobacterium breve BbIBS01 and deposited at Deutsche Sammlung von Mikroorganismen und Zellkulturen GmbH (DSMZ) under deposit number DSM 33231 (deposited on 31 Jul. 2019 by Sofar S.p.A),
(d) a bacterial strain belonging to the species Bifidobacterium breve identified as Bifidobacterium breve BbIBS02 and deposited at Deutsche Sammlung von Mikroorganismen und Zellkulturen GmbH (DSMZ) under deposit number DSM 33232 (deposited on 31 Jul. 2019 by Sofar S.p.A.),
(e) a bacterial strain belonging to the species Bifidobacterium animalis identified as Bifidobacterium animalis subsp. lactis BIIBS01 and deposited at Deutsche Sammlung von Mikroorganismen und Zellkulturen GmbH (DSMZ) under deposit number DSM 33233 (deposited on 31 Jul. 2019 by Sofar S.p.A.),
(f) a bacterial strain belonging to the species Lactobacillus plantarum identified as Lactobacillus plantarum LpIBS01 and deposited at Deutsche Sammlung von Mikroorganismen und Zellkulturen GmbH (DSMZ) under deposit number DSM 33234 (deposited on 31 Jul. 2019 by Sofar S.p.A),
(g) a bacterial strain belonging to the species Bifidobacterium bifidum identified as Bifidobacterium bifidum MIMBb23sg or BbfIBS01, or a derivative thereof, wherein said bacterial strain was deposited at Deutsche Sammlung von Mikroorganismen und Zellkulturen GmbH (DSMZ) under deposit number DSM 32708 (deposited on 04 Dec. 2017 by Sofar S.p.A);
wherein said at least one bacterial strain is for use in the modulation of at least one inflammatory/immune pathway, wherein said modulation comprises or, alternatively, consists of:
a reduction in the expression levels of at least one pro-inflammatory marker selected from the group comprising or, alternatively consisting of: interleukin IL-6, IL-8, IL-15, IL-1a, IL-1β, IL-2, IL-2 (IL-2R) receptor, iNOS (inducible nitric oxide synthase), NO (nitric oxide), TLR-4 protein, TNF-alpha and mixtures thereof, and/or
an increase in the expression levels of the anti-inflammatory marker IL-10;
wherein said at least one bacterial strain, through said modulation of inflammatory/immune pathways, is for use in a method for preventive and/or curative treatment of viral infections and/or inflammations of the respiratory system in a subject in need, preferably viral infections of the respiratory system from coronavirus, more preferably of a severe acute respiratory syndrome coronavirus (SARS-CoV, for example SARS-CoV-2 or COVID-19), and of diseases or symptoms or disorders related thereto or deriving therefrom.

The bacterial strains belonging to the species Lactobacillus paracasei are reclassified under the nomenclature Lacticaseibacillus paracasei.

The Bifidobacteria subject of the present description, such as Bifidobacterium breve BbIBS01 (DSM 33231), Bifidobacterium breve BbIBS02 (DSM 33232), Bifidobacterium animalis subsp. lactis BIIBS01 (DSM 33233), are of human origin and they are naturally found in the human intestine; while Lactobacillus plantarum LpIBS01 (DSM 33234) was isolated from the human gastrointestinal tract.

Forming an object of the present invention is a composition for use in a method for the preventive and/or curative treatment of viral infections and/or inflammations of the respiratory system in a subject in need, preferably viral infections of the respiratory system from a coronavirus, more preferably a severe acute respiratory syndrome coronavirus (e.g. SARS or COVID-19), and of diseases or symptoms or disorders related thereto or deriving therefrom, wherein said composition comprises: (i) a mixture M comprising or, alternatively, consisting of at least one belonging to the genus Lactobacillus or Bifidobacterium, preferably wherein said bacterial strain belonging to a species selected from: Lactobacillus paracasei, Lactobacillus plantarum, Bifidobacterium breve, Bifidobacterium animalis subsp. lactis and Bifidobacterium bifidum, even more preferably wherein said bacterial strain belongs to the species Lactobacillus paracasei, such as for example Lactobacillus paracasei DG® CNCM I-1572, Lactobacillus paracasei LPC-S01 DSM 26760; and, optionally, said composition further comprises (ii) at least one acceptable pharmaceutical grade additive and/or excipient.

Forming an object of the present invention is a composition (in short, composition of the invention), comprising:
(i) a mixture M (in short, mixture M of the invention) comprising or, alternatively, consisting of at least one bacterial strain selected from the group comprising or, alternatively, consisting of: Lactobacillus paracasei DG® CNCM I-1572, Lactobacillus paracasei LPC-S01 DSM 26760, Bifidobacterium breve BbIBS01 DSM 33231, Bifidobacterium breve BbIBS02 DSM 33232, Bifidobacterium animalis subsp. lactis BIIBS01 DSM 33233, Lactobacillus plantarum LpIBS01 DSM 33234, and Bifidobacterium bifidum MIMBb23sg or BbfIBS01 DSM 32708, and a mixture thereof; and, optionally,
(ii) at least one acceptable pharmaceutical grade additive and/or excipient;
wherein said at least one composition is for use in the modulation of at least one inflammatory/immune pathway, wherein said modulation comprises or, alternatively, consists of:
a reduction in the expression levels of at least one pro-inflammatory marker selected from the group comprising or, alternatively consisting of: interleukin IL-6, IL-8, IL-15, IL-1a, IL-1β, IL-2, IL-2 (IL-2R) receptor, iNOS, NO, TLR-4 protein, TNF-alpha and mixtures thereof, and/or
an increase in the expression levels of the anti-inflammatory marker IL-10;
wherein said composition, through said modulation of inflammatory/immune pathways is for use in a method for the preventive and/or curative treatment of viral infections of the respiratory system (or respiratory tract) in a subject in need, preferably viral infections of the respiratory system from coronavirus, more preferably of a severe acute respiratory syndrome coronavirus (e.g. SARS or COVID-19), and symptoms or disorders related thereto or deriving therefrom.

Forming an object of the present invention is a method for the preventive and/or curative treatment of a viral infection and/or inflammation of the respiratory system in a subject in need, preferably viral infections and/or inflammations of the respiratory system from a coronavirus or SARS-CoV (or COVID-19), and related diseases or symptoms or disorders, through the administration (oral or inhalation) of a therapeutically effective amount of said at least one bacterial strain or composition thereof of the present invention.

In the context of the present invention, the expression “significant” reduction or increase or modulation or stimulation may for example be used to indicate “significant from a statistical point of view” or “significant from a clinical point of view”. Furthermore, “significant from a statistical point of view” (or simply “statistically significant”) may be understood to be p<0.1 or p<0.05 or p<0.01.

Preferably, the bacterial strains of the present invention, or, the compositions of the present invention are for use in significantly reducing the expression levels of the pro-inflammatory cytokine IL-6 and/or IL-8. Preferably, the bacterial strains of the present invention, or, the composition of the present invention are for use in significantly increasing (or stimulating) the expression levels of the anti-inflammatory cytokine IL-10.

For example, the bacterial strains of the present invention, or, the composition of the present invention are for use in significantly reducing the expression levels of the pro-inflammatory cytokine IL-6 and/or IL-8 and in the significantly increasing (or stimulating) the expression levels of the anti-inflammatory cytokine IL-10. Thus, the bacterial strains of the present invention or the compositions of the present invention are for use as modulators of inflammatory/immune pathways leading to a Th1/Th2 ≤ 1 ratio 1, for example, changes of the expression levels of at least one selected from IL-6, IL-8, IL-15, IL-1α, IL-1β, IL-2, (IL-2R) receptor, iNOS, NO and TLR-4 protein and TNF-alpha are measured for Th1 while changes of the expression levels of IL-10 or another suitable Th2 are measured for Th2.

In the case of viral infections, it has been observed that the Th1/Th2 ratio is more shifted toward Th1, which, once induced, results in the release of pro-inflammatory cytokines (for example IL-6) and a reduction of anti-inflammatory cytokines (for example IL-10). Therefore, it is necessary to suppress the Th1 response as soon as possible and to balance Th1/Th2, so that inflammation and the ensuing tissue damage can be controlled.

Symptoms or disorders deriving from or related to said viral infection of the respiratory tract, preferably from a coronavirus (e.g. SARS-CoV, SARS-CoV-2, SARS-CoV-like), or severe acute respiratory syndrome coronavirus (e.g. SARS or COVID-19) may be: respiratory complications, asthma, chronic obstructive pulmonary disease (COPD), bronchitis, emphysema, cystic fibrosis, cough, pertussis, pneumonia, pleurisy, bronchiolitis, cold, sinusitis, rhinitis, tracheitis, pharyngitis, laryngitis, acute laryngotracheobronchitis, epiglottitis, bronchiectasis, difficulty breathing, fever, fatigue, muscle ache and/or pain, nasal congestion, runny nose, sore throat, gastrointestinal symptoms such as for example nausea and diarrhoea, renal insufficiency, loss of appetite and/or general feeling of malaise.

In an embodiment, said composition of the invention does not comprise lactoferrin or a derivative thereof (e.g. apolactoferrin, lactoferricin), and/or N-acetylcysteine or a salt thereof, and/or hyaluronic acid or a salt thereof.

The aforementioned bacterial strains present in the mixture M of the composition of the invention may be viable bacterial strains (or probiotics) derivatives of bacterial strains such as paraprobiotics, postbiotics lysates, tyndallized and/or inactivated, obtained according to methods and equipment known to the man skilled in the art.

“Probiotics” are live and viable micro-organisms (i.e. bacterial strains) which, when administered in adequate amount, confer benefits to the health of the host; the term “probiotics” refers to micro-organisms present in or added to food (FAO and WHO definition).

In the context of the present invention, the term “derivative” of a bacterial strain (or “derivative” of a viable bacterial strain) is used to indicate the bacterial strain tyndallized or sonicated or inactivated using other techniques known to the man skilled in the art (for example using gamma rays), or lysates of the bacterial strain or extracts of the bacterial strain (in short, paraprobiotics) or any derivative and/or component of the bacterial strain, preferably exopolysaccharide, parietal fraction, metabolites or metabolic bioproducts generated by the bacterial strain (in short, postbiotics) and/or any other product derived from the bacterial strain. Preferably, the term “derivative” of the bacterial strains of the present invention is used to indicate the bacterial strain tyndallized or inactivated (for example using gamma rays).

In other words, the term “derivative” of a probiotic viable bacterial strain, in the context of the present invention, is substantially used to indicate a paraprobiotic or a postbiotic.

In the context of the present invention, the term “paraprobiotics” is used to indicate bacterial cells (i.e. intact or broken) that are non-viable (i.e., without the ability to replicate) or crude cell extracts which, when administered in an adequate amount, confer a benefit to the health of the host (similarly to the viable bacterial strain from which they derive). Examples of paraprobiotics are heat inactivated bacterial strains (for example tyndallized bacterial strains), sonication (ultrasonic), gamma irradiation (gamma rays), or lysates of bacterial strains or extracts of bacterial strains.

In the context of the present invention, the term “postbiotics” is used to indicate any substance released or produced by means of the metabolic activity of the probiotic viable bacteria strain, wherein said postbiotics, when administered in an adequate amount, confer a benefit to the health of the host (similarly to the viable bacterial strain from which they derive). Examples of postbiotics are exopolysaccharides, parietal fractions, metabolites or metabolic bioproducts.

According to a preferred aspect, the mixture M of the composition of the invention comprises or, alternatively, consists of a bacterial strain belonging to the species Lactobacillus paracasei.

Preferably, the mixture M of the composition of the invention comprises or, alternatively, consists of a Lactobacillus paracasei DG® CNCM I-1572 strain.

Alternatively, the mixture M of the composition of the invention may comprise or, alternatively, consist of a Lactobacillus paracasei DG® CNCM I-1572 strain and a Lactobacillus paracasei LPC-S01 DSM 26760 strain.

The strain Lactobacillus paracasei DG® CNCM I-1572 and/or the strain Lactobacillus paracasei LPC-S01 DSM 26760 have shown to have the ideal characteristics to boost the gut microbiota and modulate the suitable inflammatory/immune pathways so as to be effective in a method for the treatment of viral infections in subjects in need, preferably viral infections of the respiratory system from coronavirus, more preferably a severe acute respiratory syndrome coronavirus (SARS or COVID-19).

In particular, the strain L. paracasei DG® CNCM I-1572 and/or L. paracasei LPC-S01 DSM 26760 can be used in the treatment of viral diseases of the present invention given that they can reduce the expression levels of interleukins IL-6, IL-8, IL-15, of the family IL-1 (e.g. IL-1α and/or IL-1β), IL-2, (IL-2R) receptor, iNOS (thus controlling the release of NO), and/or of the TLR-4 protein, TNF-alpha; furthermore, the strain L. paracasei DG® CNCM I-1572 and/or L. paracasei LPC-S01 DSM 26760 can stimulate the expression levels of interleukin IL-10.

Furthermore, it has been observed that:
L. casei DG® CNCM I-1572 and/or L. paracasei LPC-S01 DSM 26760 survive during gastrointestinal transit and reach the gut alive and viable. L. casei DG® CNCM I-1572 was tested in recovery studies; recovery studies are the only evidence that the strain is actually capable of passing through the gastrointestinal tract and colonising the intestine in humans;
L. casei DG® CNCM 1-1572 is capable of positively modulating the structure/function of the gut microbiota, thus favouring the gut microbiota balance by statistically significantly increasing Lactobacilli (which are reduced in patients with coronavirus infection) and by reducing pathogenic and/or potentially pathogenic bacterial populations;
L. casei DG® CNCM I-1572 and/or L. paracasei LPC-S01 DSM 26760 are capable of positively modulating inflammatory/immune pathways, inducing a statistically significant reduction in IL-1α, IL-15, IL-6 and IL-8 and a statistically significant increase in IL-10. For L. casei DG® CNCM I-1572 such effect could be attributable to the presence of its peculiar EPS (exopolysaccharide), which covers the bacterium like a sort of natural “microencapsulation”;
L. casei DG® CNCM I-1572 induces a statistically significant increase in faecal levels of short-chain fatty acids, such as acetate and especially butyrate.

Further examples of the composition of the invention, depending on the change of the mixture M, are reported below.

Said mixture M of the invention may comprise or, alternatively, consist of:(a) L. paracasei DG® CNCM I-1572, or a derivative thereof, and furthermore a mixture of the bacterial strains comprising (c) B. breve BblBS01 DSM 33231, (d) B. breve BblBS02 DSM 33232, (e) B. animalis subsp. lactis BIIBS01 DSM 33233, (f) L. plantarum LplBS01 DSM 33234 and, optionally, (g) B. bifidum MIMBb23sg = BbflBS01 DSM 32708, or derivatives thereof.

Said mixture M of the invention may comprise or, alternatively, consist of: (a) L. paracasei DG® CNCM I-1572, or a derivative thereof, and (b) L. paracasei LPC-S01 DSM 26760, or a derivative thereof, and furthermore a mixture of the bacterial strains comprising (c) B. breve BblBS01 DSM 33231, (d) B. breve BblBS02 DSM 33232, (e) B. animalis subsp. lactis BIIBS01 DSM 33233, (f) L. plantarum LplBS01 DSM 33234 and, optionally, (g) B. bifidum MIMBb23sg = BbflBS01 DSM 32708, or derivatives thereof.

Said mixture M of the invention may comprise or, alternatively, consist of: (a) L. paracasei DG® CNCM I-1572, or a derivative thereof, and furthermore at least one bacterial strain (or a derivative thereof) selected from: (c) B. breve BblBS01 DSM 33231, (d) B. breve BblBS02 DSM 33232, (e) B. animalis subsp. lactis BIIBS01 DSM 33233, (f) L. plantarum LplBS01 DSM 33234 and (g) B. bifidum MIMBb23sg = BbflBS01 DSM 32708, and a mixture thereof.

Said mixture M of the invention may comprise or, alternatively, consist of: (a) L. paracasei DG® CNCM I-1572, or a derivative thereof, and (b) L. paracasei LPC-S01 DSM 26760, or a derivative thereof, and furthermore at least one bacterial strain (or a derivative thereof) selected from: (c) B. breve BblBS01 DSM 33231, (d) B. breve BblBS02 DSM 33232, (e) B. animalis subsp. lactis BIIBS01 DSM 33233, (f) L. plantarum LplBS01 DSM 33234 and (g) B. bifidum MIMBb23sg = BbflBS01 DSM 32708, and a mixture thereof.

The composition of the invention comprising said mixture M according to any embodiment of the present invention, preferably comprising or, alternatively, consisting of the strain L. paracasei DG® CNCM I-1572 and/or the strain L. paracasei LPC-S01 DSM 26760, furthermore, it may comprise at least one prebiotic, for example selected from: inulin, fructooligosaccharide (FOS), galacto-oligosaccharide (GOS), guar gum and mixtures thereof; preferably inulin.

For example, the composition of the invention may comprise or, alternatively, consist of the strain Lactobacillus paracasei DG® CNCM I-1572 and/or the strain Lactobacillus paracasei LPC-S01 DSM 26760 and inulin.

Furthermore, the composition of the invention comprising said mixture M according to any embodiment of the present invention, preferably comprising or, alternatively, consisting of the strain L. paracasei DG® CNCM I-1572 and/or the strain L. paracasei LPC-S01 DSM 26760, and optionally at least one prebiotic (e.g. inulin), may further comprise:
at least one vitamin, such as for example those of group B, vitamin C, vitamin D, vitamin A and vitamin E; and/or
antioxidant substances, such as for example glutathione, polyphenols such as resveratrol and trans-resveratrol, coenzyme Q10, astaxanthin, lycopene; and/or
plant extracts (botanicals), such as Echinacea, Uncaria tomentosa, fermented papaya, berries e ginger ( Zingiber officinale); and/or
minerals, such as for example zinc, selenium, magnesium, iron, potassium, copper; and/or
amino acids, such as glutamine, arginine, tryptophan; and/or
omega-3 fatty acids.

The compositions of the present invention, according to any one of the described embodiments, can be formulated for oral use, for nasal inhalation (e.g. spray or drops), for oral inhalation (e.g. spray, dry powders for inhalation). In the context of the present invention, the term for oral use is used to indicate both oral (or gastroenteric) administration and sublingual (or buccal) administration.

When the composition of the present invention is formulated for oral use, it can be in solid form selected from: tablets, chewable tablets, buccal tablets, granules, flakes, soluble powder or granules, oral soluble powder or granules, capsules; or, alternatively, in liquid form selected from: solutions, suspensions, dispersions, emulsions, liquid which can be dispensed in spray form, syrups; or, alternatively, in semiliquid form selected from: soft-gel, gel; preferably the composition of the invention is for oral use in solid or liquid form.

Advantageously, said at least one bacterial strain or mixture of bacterial strains are present in the composition of the invention (or each bacterial strain is present) in a concentration comprised in the range from 10×10⁶ CFU to 10×10¹² CFU, preferably from 10×10⁸ CFU to 10×10¹⁰ CFU, more preferably in a concentration of about 10×10⁸ CFU or 10×10⁹ CFU, with respect to the daily dose (CFU: Colony forming Unit).

The aforementioned daily doses can be administered to the subject in need in a single dose (one dose) or in repeated doses, for example two, three or four daily doses.

The compositions of the invention according to any one of the described embodiments may be for use as adjuvants of further approaches for treating viral infections of the respiratory system, preferably of a severe acute respiratory syndrome coronavirus (e.g. COVID-19).

The composition of the invention, comprising said mixture M according to any one of the embodiments of the present invention, may further comprise said at least one pharmaceutical or food grade additive and/or excipient, i.e. a substance devoid of therapeutic activity suitable for pharmaceutical or food use. In the context of the present invention the additives and/or excipients acceptable for pharmaceutical or food use comprise all ancillary substances known to the man skilled in the art for the preparation of compositions in solid, semi-solid or liquid form, such as for example diluents, solvents (including water, glycerine, ethyl alcohol), solubilisers, acidifiers, thickeners, sweeteners, flavour-enhancement agents, colouring agents, lubricants, surfactants, preservatives, stabilisers, pH stabilising buffers and mixtures thereof.

Unless specified otherwise, the expression composition or mixture or other comprising a component at an amount “comprised in a range from x to y” is used to indicate that said component can be present in the composition or mixture or other at all the amounts present in said range, even though not specified, extremes of the range comprised.

Unless specified otherwise, the indication that a composition or mixture “comprises” one or more components or substances means that other components or substances can be present besides the one, or the ones, indicated specifically.

In the context of the present invention, the expression “treatment method” is used to indicate an intervention on a subject in need, comprising the administration of a therapeutically effective amount (according to a man skilled in the art) of a bacterial strain or composition or mixture of substances with the aim of eliminating, reducing/decreasing or preventing a disease or ailment and symptoms or disorders thereof.

In the context of the present invention, the term “subject/s” is used to indicate human or animal subjects, preferably mammals (e.g. pets such as dogs, cats, horses, sheep or cattle). Preferably, the compositions of the invention are for use in treatment methods for human subjects.

### EXAMPLES

An example of a composition according to the invention is the commercial product Enterolactis® (trademark registered by Sofar spa, Italy) comprising the bacterial strain Lactobacillus paracasei DG® CNCM I-1572.

A further example of composition according to the invention is the commercial product Enterolactis® Duo (trademark registered on behalf of Sofar spa, Italy) comprising the bacterial strain Lactobacillus paracasei DG® CNCM I-1572 and inulin (Enterolactis® Duo).

Embodiments of the present invention FR -Ans are reported below.

FR-A1. A composition for use in a method of treating a viral infection of the respiratory system in a subject in need, and of diseases or symptoms related thereto, wherein said composition comprises: (i) a mixture (M) comprising or, alternatively, consisting of at least one bacterial strain selected from: Lactobacillus paracasei DG® CNCM I-1572, Lactobacillus paracasei LPC-S01 DSM 26760, Bifidobacterium breve BblBS01 DSM 33231, Bifidobacterium breve BblBS02 DSM 33232, Bifidobacterium animalis subsp. lactis BIIBS01 DSM 33233, Lactobacillus plantarum LplBS01 DSM 33234, Bifidobacterium bifidum MIMBb23sg = BbflBS01 DSM 32708, and a mixture thereof; and, optionally, (ii) at least one acceptable pharmaceutical grade additive and/or excipient.

FR-A2. The composition for use according to FR-A1, wherein said composition is for use in a method of treating a viral infection of the respiratory system from a coronavirus, preferably SARS-CoV-2 virus, in a subject in need, and of diseases or symptoms related thereto.

FR-A3. The composition for use according to FR-A1 or 2, wherein said composition is for use in a method for the treatment of a severe acute respiratory syndrome coronavirus (SARS or COVID-19) in a subject in need, and of diseases or symptoms related thereto.

FR-A4. The composition for use according to any one of FR-A1-3, wherein said composition modulates at least one inflammatory/immune pathway through:
a reduction in the expression levels of at least one pro-inflammatory marker selected from interleukin IL-6 and/or interleukin IL-8; and/or
an increase in the expression levels of the anti-inflammatory marker IL-10.

FR-A5. The composition for use according to any one of FR-A1-4, wherein said composition modulates at least one inflammatory/immune pathway through:
a reduction in the expression levels of at least one pro-inflammatory marker selected from the group comprising or, alternatively, consisting of: interleukin IL-6, IL-8, IL-15, IL-1α, IL-1β, IL-2, IL-2 (IL-2R) receptor, iNOS, NO, TLR-4 protein, TNF-alpha and mixtures thereof, and/or
an increase in the expression levels of the anti-inflammatory marker IL-10.

FR-A6. The composition for use according to any one of FR-A1-5, wherein said related diseases or symptoms are selected from: respiratory complications, asthma, chronic obstructive pulmonary disease (COPD), bronchitis, emphysema, cystic fibrosis, cough, pertussis, pneumonia, pleuritis, bronchiolitis, cold, sinusitis, rhinitis, tracheitis, pharyngitis, laryngitis, acute laryngotracheobronchitis, epiglottitis, bronchiectasis, difficulty breathing, fever, fatigue, muscle ache and/or pain, nasal congestion, runny nose, sore throat, nausea, diarrhoea, renal insufficiency and loss of appetite.

FR-A7. The composition for use according to any one of FR-A1-6, wherein said mixture (M) consists of a Lactobacillus paracasei DG® CNCM I-1572 strain.

FR-A8. The composition for use according to any one of FR-A1-6, wherein said mixture (M) consists of a Lactobacillus paracasei DG® CNCM I-1572 strain and a Lactobacillus paracasei LPC-S01 DSM 26760 strain.

FR-A9. The composition for use according to any one of FR-A1-8, wherein said composition further comprises at least one prebiotic; preferably said at least one prebiotic is selected from: inulin, fructooligosaccharide (FOS), galacto-oligosaccharide (GOS), guar gum and mixtures thereof, preferably inulin.

FR-A10. A bacterial strain for use in a treatment method according to any one of the FR-A1-6, wherein said bacterial strain is selected from the group consisting of: Lactobacillus paracasei DG® CNCM I-1572; Lactobacillus paracasei LPC-S01 DSM 26760; Bifidobacterium breve BblBS01 DSM 33231; Bifidobacterium breve BblBS02 DSM 33232; Bifidobacterium animalis subsp. lactis BIIBS01 DSM 33233; Lactobacillus plantarum LplBS01 DSM 33234; and Bifidobacterium bifidum MIMBb23sg (= BbflBS01) DSM 32708.

Preferred embodiments of the present invention FR -Bns are reported below.

FR-B1. A composition for use in a method of treating a viral infection of the respiratory system caused by a virus belonging to the species severe acute respiratory syndrome coronavirus (SARS-CoV) in a subject in need, such as a severe acute respiratory syndrome coronavirus and of diseases or symptoms related thereto, wherein said composition comprises:
(i) a mixture (M) comprising or, alternatively, consisting of at least one bacterial strain belonging to the species Lactobacillus paracasei; and, optionally,
(ii) at least one acceptable pharmaceutical grade additive and/or excipient.

FR-B2. The composition for use according to FR-B1, wherein said at least one bacterial strain belonging to the species Lactobacillus paracasei is selected from the group comprising or, alternatively, consisting of:
a bacterial strain belonging to the species Lactobacillus paracasei identified as Lactobacillus paracasei DG® and deposited at the National Collection of Cultures of Microorganisms of the Pasteur Institute in Paris under the accession number CNCM I-1572,
a bacterial strain belonging to the species Lactobacillus paracasei identified as Lactobacillus paracasei LPC-S01 and deposited at Deutsche Sammlung von Mikroorganismen und Zellkulturen GmbH (DSMZ) under the accession number DSM 26760, and a mixture thereof.

FR-B3. The composition for use according to FR-B1 or FR-B2, wherein said coronavirus is a virus of the strain severe acute respiratory syndrome coronavirus-2 (SARS-CoV-2) responsible for the disease COVID-2019.

FR-B4. The composition for use according to any one of FR-B1- FR-B3, wherein said composition is for oral use.

FR-B5. The composition for use according to any one of FR-B1- FR-B4, wherein said mixture (M) consists of a Lactobacillus paracasei DG® CNCM I-1572 strain.

FR-B6. The composition for use according to any one of FR-B1- FR-B4, wherein said mixture (M) consists of a Lactobacillus paracasei DG® CNCM I-1572 strain and a Lactobacillus paracasei LPC-S01 DSM 26760 strain.

FR-B7. The composition for use according to any one of FR-B1 -FR-B6 wherein said mixture M further comprises at least one further bacterial strain selected from the group comprising or, alternatively, consisting of:
a bacterial strain belonging to the species Bifidobacterium breve identified as Bifidobacterium breve BblBS01 and deposited at Deutsche Sammlung von Mikroorganismen und Zellkulturen GmbH (DSMZ) under deposit number DSM 33231,
a bacterial strain belonging to the species Bifidobacterium breve identified as Bifidobacterium breve BblBS02 and deposited at Deutsche Sammlung von Mikroorganismen und Zellkulturen GmbH (DSMZ) under deposit number DSM 33232,
a bacterial strain belonging to the species Bifidobacterium animalis identified as Bifidobacterium animalis subsp. lactis BIIBS01 and deposited at Deutsche Sammlung von Mikroorganismen und Zellkulturen GmbH (DSMZ) under deposit number DSM 33233,
a bacterial strain belonging to the species Lactobacillus plantarum identified as Lactobacillus plantarum LplBS01 and deposited at Deutsche Sammlung von Mikroorganismen und Zellkulturen GmbH (DSMZ) under deposit number DSM 33234,
a bacterial strain belonging to the species Bifidobacterium bifidum identified as Bifidobacterium bifidum MIMBb23sg = BbflBS01 and deposited at Deutsche Sammlung von Mikroorganismen und Zellkulturen GmbH (DSMZ) under deposit number DSM 32708, and
a mixture thereof.

FR-B8. The composition for use according to any one of FR-B1 - FR-B7, wherein said mixture (M) comprises or, alternatively, consists of a Lactobacillus paracasei DG® CNCM I-1572 strain and furthermore a further bacterial strain selected from the group comprising or, alternatively, consisting of: Bifidobacterium breve BblBS01 DSM 33231, Bifidobacterium breve BblBS02 DSM 33232, Bifidobacterium animalis subsp. lactis BIIBS01 DSM 33233, Lactobacillus plantarum LplBS01 DSM 33234, Bifidobacterium bifidum MIMBb23sg (= BbflBS01)DSM 32708 and a mixture thereof.

FR-B9. The composition for use according to any one of FR-B1 - FR-B8, wherein said at least one bacterial strain is a probiotic viable bacterial strain or a paraprobiotic or a postbiotic.

FR-B10. The composition for use according to any one of FR-B1 - FR-B9, wherein said composition further comprises at least one prebiotic; preferably said at least one prebiotic is selected from: inulin, fructooligosaccharide (FOS), galacto-oligosaccharide (GOS), guar gum and mixtures thereof, preferably inulin.

FR-B11. The composition for use according to any one of FR-B1 - FR-B10, wherein said related diseases or symptoms are selected from: respiratory complications, asthma, chronic obstructive pulmonary disease (COPD), bronchitis, emphysema, cystic fibrosis, cough, pertussis, pneumonia, pleuritis, bronchiolitis, cold, sinusitis, rhinitis, tracheitis, pharyngitis, laryngitis, acute laryngotracheobronchitis, epiglottitis, bronchiectasis, difficulty breathing, fever, fatigue, muscle ache and/or pain, nasal congestion, runny nose, sore throat, nausea, diarrhoea, renal insufficiency and loss of appetite.

FR-B12. A bacterial strain for use in a treatment method according to any one of FR-B1 - FR-B11, wherein said bacterial strain is selected from the group consisting of:
Lactobacillus paracasei DG® deposited at the National Collection of Cultures of Microorganisms of the Pasteur Institute in Paris under the accession number CNCM I-1572;
Lactobacillus paracasei LPC-S01 deposited at Deutsche Sammlung von Mikroorganismen und Zellkulturen GmbH (DSMZ) under the accession number DSM 26760;
Bifidobacterium breve BblBS01 deposited at Deutsche Sammlung von Mikroorganismen und Zellkulturen GmbH (DSMZ) under deposit number DSM 33231;
Bifidobacterium breve BblBS02 deposited at Deutsche Sammlung von Mikroorganismen und Zellkulturen GmbH (DSMZ) under deposit number DSM 33232;
Bifidobacterium animalis subsp. lactis BIIBS01 deposited at Deutsche Sammlung von Mikroorganismen und Zellkulturen GmbH (DSMZ) under deposit number DSM 33233;
Lactobacillus plantarum LplBS01 deposited at Deutsche Sammlung von Mikroorganismen und Zellkulturen GmbH (DSMZ) under deposit number DSM 33234; and
Bifidobacterium bifidum MIMBb23sg = BbflBS01 deposited at Deutsche Sammlung von Mikroorganismen und Zellkulturen GmbH (DSMZ) under deposit number DSM 32708.

### Experimental Part

### 1. Purpose

The Applicant conducted in vitro studies in order to evaluate the ability of compositions according to the present invention comprising at least one bacterial strain belonging to the species Lactobacillus paracasei, such as Lactobacillus paracasei DG® (CNCM I-1572) and/or Lactobacillus paracasei LPC-S01 (DSM 26760), to stimulate the innate antiviral immune response in a subject so as to combat a viral infection, particularly a viral infection of the respiratory tract caused by the SARS-CoV-2 virus (COVID-19).

In detail, the following were evaluated in vitro:
(1) the ability of compositions according to the present invention to boost antiviral responses in intestinal epithelial cells (antiviral immunomodulatory effect); and
(2) the ability of compositions according to the present invention to influence SARS-CoV-2 infection in human intestinal epithelial cells (SARS-CoV-2 infection in vitro model).

### 2. Material

### 2.1. Cells, Viruses, Bacterial Strains and Reagents

The Caco-2 human colon adenocarcinoma cell line (ATCC® HTB-37™) and
the Vero E6 monkey kidney epithelial cell line (ATCC® CRL-1586™)
were cultured in DMEM medium supplemented with 10% (v/v) FBS, 1% (v/v) sodium pyruvate and 1% (v/v) penicillin/streptomycin (all from Gibco-Thermo Fisher Scientific, Waltham, USA) at 37° C. in a humidified incubator containing 5% CO₂.

Probiotic bacterial strains, such as:
Lactobacillus rhamnosus GG (ATCC 53103), new nomenclature Lacticaseibacillus rhamnosus;
Lactobacillus paracasei DG® (CNCM I-1572; L. casei DG®; Enterolactis®, SOFAR SpA), new nomenclature Lacticaseibacillus paracasei;
L. Lactobacillus paracasei LPC-S01 (DSM 26760) new nomenclature Lacticaseibacillus paracasei; and
Bifidobacterium bifidum MIMBb23sg (or BbflBS01)(DSM 32708).

The strains were cultured on MRS plates (DeMan Rogosa Sharpe, Difco, BD) and incubated for 72 hours at 37° C. under anaerobic conditions.

The GG (ATCC 53103) strain was purchased from the ATCC collection, while the DG® (CNCM I-1572), LPC-S01 (DSM 26760) and MIMBb23sg (DSM 32708) strains were supplied by Sofar SpA (Milan, Italy).

A sterile DMEM with a high glucose content supplemented with 20% glycerol was inserted as a control test.

### 2.2. Preparation and Titration of Viral Stocks

SARS-CoV-2 was isolated from a patient at the Microbiology Unit, University Hospital of Padua. The viral strain was propagated in Vero E6 cells and characterised by the sequencing of the entire genome. The viral titre was determined using the plaque test method. In short, VERO E6 confluent cells in 24-well plates (Costar, Merck, Italy) were inoculated with 10-fold serial dilutions of the virus stock for 1 hour. Then, the growth medium was removed and the cells incubated with fresh medium containing carboxymethylcellulose (CMC, Merck). The cells were fixed 72 hours p.i. with 5% w/v formaldehyde (Merck) and stained with crystal violet (Merck). The viral titre was measured as a plaque-forming unit (PFU/mL) based on the plaques formed in the cell culture after infection. All infection experiments were conducted in a biosafety level 3 (BSL-3) laboratory at the Department of molecular Medicine, University of Padua, Padua, Italy.

### 2.3. Preparation of Bacterial Strains

### 2.3.1. Viable Cells

Broth cultures were prepared in De Man, Rogosa, Sharpe (MRS) broth with incubation for 18 hours at 37° C. under anaerobic conditions. After incubation, the strains were centrifuged for 10 minutes at 3000 rpm and the cell pellets were washed twice with sterile distilled water. The optical density at 600 nm (OD600) of the washed cultures was adjusted to 0.3 to reach 2.5 × 10⁶ CFU in 20 µl volume. The standardised washed cultures were diluted in series for viable count and centrifuged for ten minutes at 3000 rpm. The pellets were resuspended in sterile DMEM medium (Gibco-Thermo Fisher Scientific, Waltham, USA) supplemented with 20% glycerol (Merck).

### 3. Methods

### 3.1. Caco-2 Cell Culture and Experimental Design

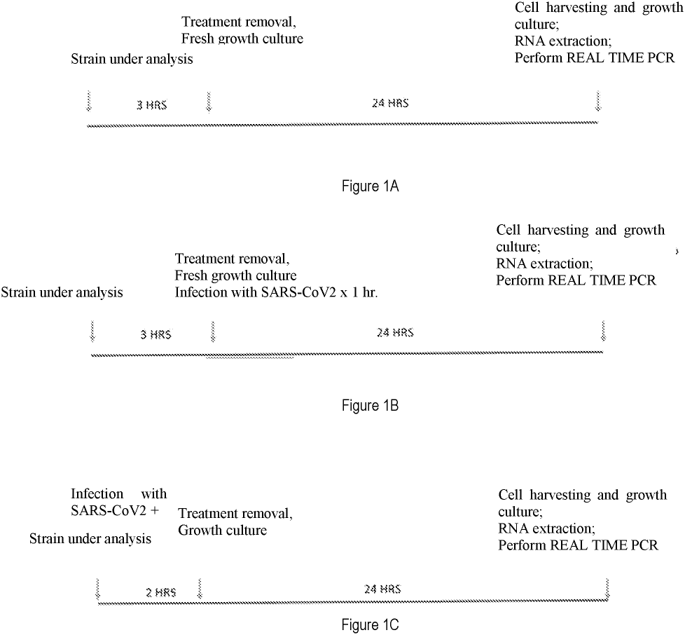

Caco-2 cells were seeded in 12-well plates (2 × 10⁵ cells/mL). After reaching confluence, the cells were washed in 1x PBS (Gibco-Thermo Fisher Scientific, Waltham, USA) and incubated in antibiotic-free medium (AFM) or subjected to one of the following treatments (FIG. 1).

Treatment with probiotics alone in the absence of SARS-CoV-2 virus (FIG. 1A).

Pre-treatment with probiotics with respect to treatment with SARS-CoV-2 virus (FIG. 1B).

The confluent Caco-2 cells were supplemented with the bacterial strain (viable; MOI1:10).

After 3 hours, the cells were washed in 1x PBS (Gibco-Thermo Fisher Scientific, Waltham, USA) and incubated with fresh medium supplemented with antibiotics (penicillin / streptomycin), then infected with SARS-CoV-2 (MOI 1:2) for 1 hour. 24 hours p.i. cells were harvested for RNA extraction.

Co-treatment with probiotics and of SARS-CoV-2 virus (FIG. 1C).

The confluent Caco-2 cells were supplemented with the bacterial strain (viable; MOI 1:10) together with SARS-CoV-2 (MOI 1:2). After 3 hours, the cells were washed and incubated with fresh medium for another 24 hours before harvesting for RNA extraction.

### 3.2. RNA Extraction and Real Time PCR

Total RNA was isolated using the E.Z.N.A.® Total RNA Kit I (Omega Bio-Tek, tebu-bio, Italy) following the manufacturer’s instructions. The contaminant DNA was removed by incubation with RNase-free DNase I sets (Omega Bio-Tek). Complementary DNA synthesis and amplification were conducted using iTaqTM Universal Probes One-Step Kit (Bio-Rad, Milan, Italy) according to the manufacturer’s recommendations in an ABI PRISM 7000 Sequence Detection (Applied Biosystems) system. The target gene expression was normalised to the expression of the reference gene GAPDH.

The data are presented as a mean fold change on the control.

### 3.3. Statistical Analysis

The data are shown as mean +/- SD (SD: Standard deviation). Statistical analysis was conducted using GraphPad Prism Software 6.0 software (GraphPad Software Inc., La Jolla, USA). Comparisons were conducted using the two-tailed student’s t test. The differences were deemed significant with p <0.05 (the following keys are shown in the figures: * p<0.05; ** p<0.01; *** p<0.001).

### 4. Results

### 4.1. Probiotic Strains of Lacticaseibacillus Increase the Antiviral Immune Response in Vitro

The antiviral immunomodulatory effects of the probiotic strains of Lacticaseibacillus were evaluated in vitro using the Caco-2 human intestinal epithelial cells.

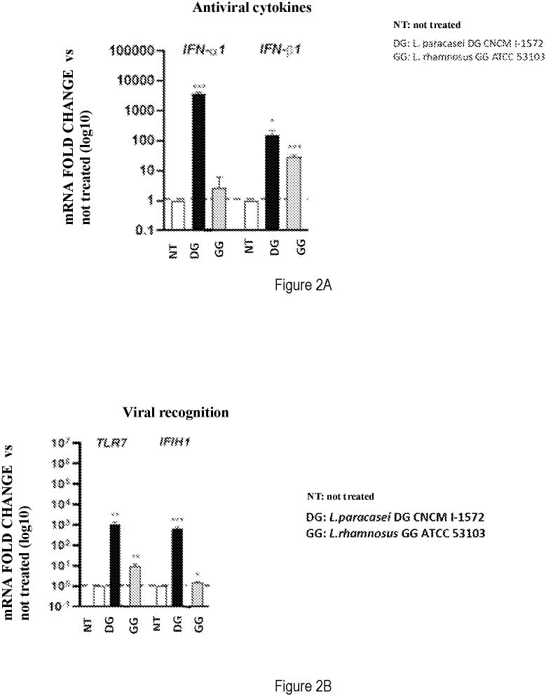

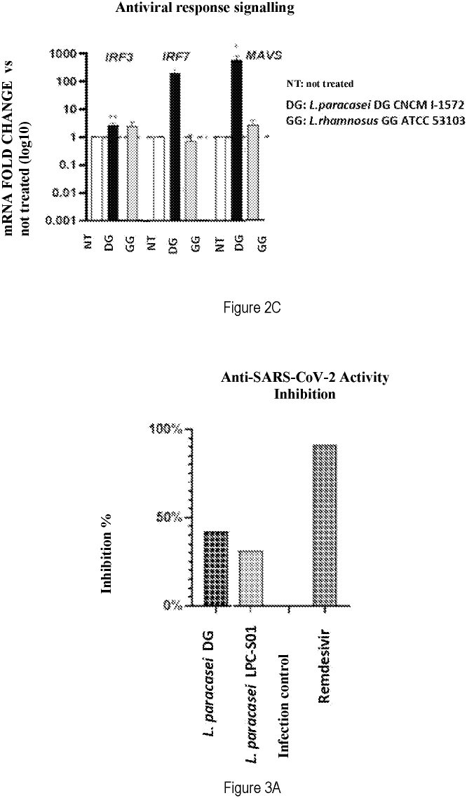

As shown in FIGS. 2A-2C, the treatment with probiotic bacterial strains according to the present invention, such as Lactobacillus paracasei DG® (CNCM I-1572) induced significant changes in the expression profile of several genes involved in the antiviral immune response.

The level of the antiviral cytokine interferon alpha (IFN-alpha1) was significantly improved by the L. paracasei DG® (CNCM I-1572) strain, and a tendency toward upregulation of interferon beta (IFN-beta1) (FIG. 2A).

Furthermore, L. paracasei DG® (CNCM I-1572) significantly increased the expression of TLR7, a pattern recognition receptor involved in the detection of RNA virus, of IFIH1, the gene encoding for MDA5 which is a molecular sensor of viral RNA, and also of IRF3, IRF7 and MAVS, which participate in the antiviral signalling pathways of response (FIGS. 2B and 2C).

Based on these results of the antiviral immune boosting activity, the strain L. paracasei DG® (CNCM I-1572) was selected for further testing.

### 4.2. Inhibitory Effect of the Probiotic Strains of Lactobacillus Paracasei on SARS-CoV-2 Replication in Vitro

To evaluate the antiviral activity of several probiotic strains against SARS-CoV-2, an infection assay for SARS-CoV-2 in Caco-2 cells was conducted.

Prior to virus infection, the cells were pre-treated with probiotic strains for 3 hours and then infected with SARS-CoV-2 for 1 hour (FIG. 1B). The expression level of virus-specific genes encoding RNA-dependent RNA polymerase (RdRp) and E gene (CoVE), which are critical for the replication and assembly of SARS-CoV-2, was analysed from the total RNA obtained from the harvested cells.

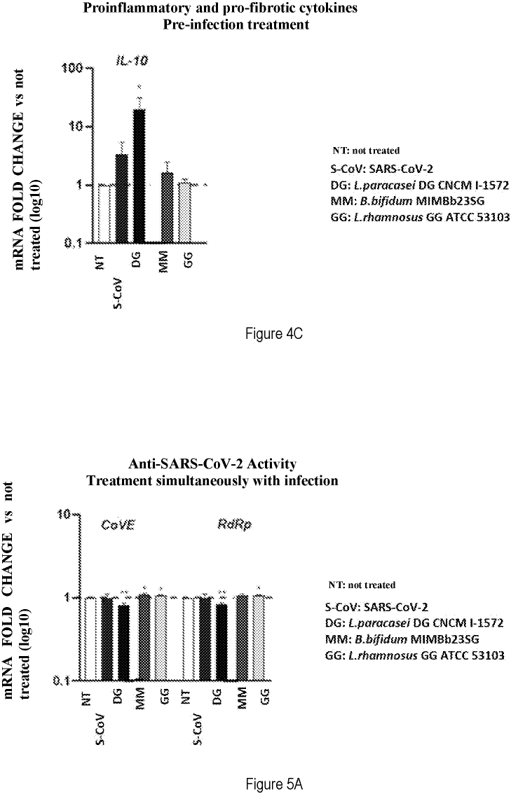

It was observed that the expression of both genes (RdRp and CoVE) was significantly reduced in the Caco-2 cells treated with L. paracasei DG® (CNCM I-1572) (FIG. 4A), indicating that pre-treatment with probiotic strains could inhibit SARS-CoV-2 replication in vitro.

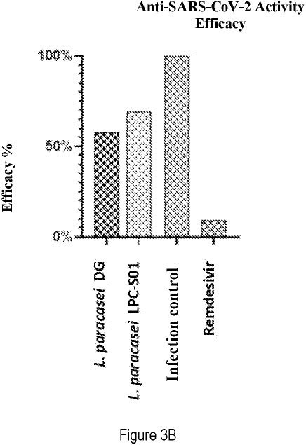

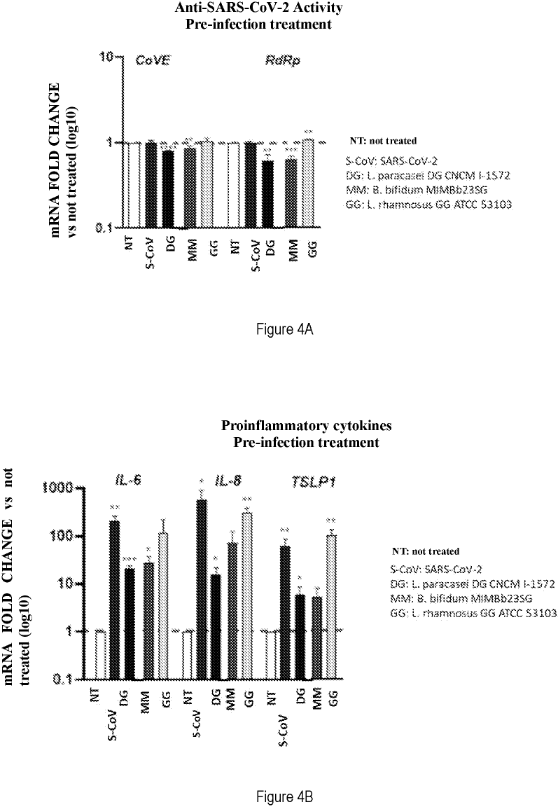

Furthermore, the SARS-CoV-2 titre was also evaluated on the harvested supernatants: pre-treatment with L. paracasei DG® (CNCM I-1572) determined 45.8% inhibition of SARS-CoV-2 infection and compared with Remdesivir, a broad-spectrum antiviral drug (Gilead Sciences) (FIG. 3A, value expressed as inhibition and FIG. 3B, value expressed as efficacy).

### 4.3. Pre-Treatment with the Bacterial Strains of the Present Invention Protects Against the Inflammatory Response Triggered by SARS-CoV-2 in Vitro

It is known that proinflammatory and profibrotic cytokines are increased by SARS-CoV-2 infection and in the most severe cases the prognosis of patients can be considerably worsened by the hyperproduction of proinflammatory cytokines.

To determine whether pre-treatment with probiotic strains can protect against the inflammatory response triggered by SARS-CoV-2 infection in vitro, the inflammatory and anti-inflammatory cytokine expression profile of SARS-CoV-2 infected Caco-2 cells pre-treated or not-treated with strains of L. paracasei were tested (FIG. 4). Transcription levels of all measured cytokines tended to be upregulated following SARS-CoV-2 infection (data not shown).

In particular, pre-treatment of the Caco-2 cells infected with the strain L. paracasei DG® (CNCM I-1572) significantly reduced the mRNA expression levels of the IL6, IL8 and TSLP1 genes and increased the mRNA expression levels of the IL10 genes (FIGS. 4A-4C), with respect to the control and with respect to Lactobacillus rhamnosus GG (ATCC 53103).

Furthermore, it should also be observed that pre-treatment of the Caco-2 cells infected the strain B bifidum MIMBb23sg (= BbflBS01)DSM 32708 significantly reduced the mRNA expression levels of the IL6, IL8 and TSLP1 genes and the expression level of virus-specific genes encoding RNA-dependent RNA polymerase (RdRp) and gene E (CoVE) (FIGS. 4A-4C), with respect to the control and with respect to Lactobacillus rhamnosus GG (ATCC 53103).

### 4.4. Co-Treatment with the Bacterial Strains of the Present Invention Protects Against the Inflammatory Response Triggered by SARS-CoV-2 in Vitro

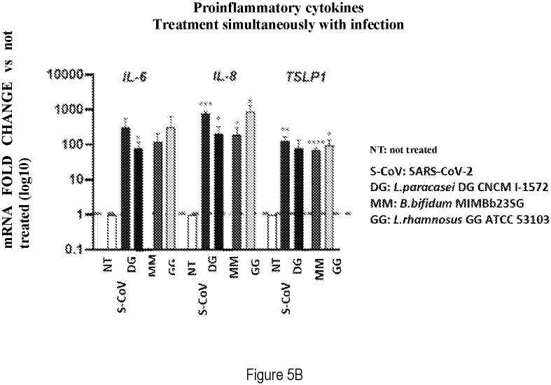

Results similar to what is reported in paragraph 4.3 were obtained in the Caco-2 cell co-treatment study (FIG. 1C and FIGS. 5A-B).

### 5. Conclusion

The results obtained have shown that the bacterial strains according to the present invention belonging to the species Lactobacillus paracasei, preferably Lactobacillus paracasei DG® (CNCM I-1572),are capable of positively modulating the antiviral and anti-inflammatory responses, thus being useful as adjuvants in the SARS-CoV2 antiviral therapy.

In particular, the in vitro tests of the present study show both the antiviral immune system boosting activity and their ability to prevent the replication of SARS-CoV-2 by about 50%, by using compositions of the present invention.

Among the probiotic strains tested, the bacterial strains belonging to the species Lactobacillus paracasei, preferably Lactobacillus paracasei DG® (CNCM I-1572),proved to be the most promising in terms of antiviral immunomodulatory activity, capable of inducing the expression of IFN and genes involved in the antiviral response signalling pathways such as TLR7, IFIH, IRF3, IRF7 and MAVS.

Furthermore, prophylactic treatment or co-treatment in vitro with the bacterial strains belonging to the species Lactobacillus paracasei, preferably Lactobacillus paracasei DG® (CNCM I-1572),suppressed the inflammatory response triggered by SARS-CoV-2 infection in Caco-2 cells, given that the transcription levels of pro-inflammatory cytokines IL-6, IL8 and TSLP1 were reduced with respect to the control.

Thus, the preventive use of bacterial strains according to the present invention belonging to the species Lactobacillus paracasei, preferably Lactobacillus paracasei DG® (CNCM I-1572),or compositions thereof, contributes towards alleviating the excessive inflammatory response induced by SARS-CoV-2 infection.

As known in literature, the bacterial strain Lactobacillus paracasei DG® (CNCM I-1572) is a probiotic strain that has been shown to survive gastrointestinal transit, both in adults and children.

In the present study, the bacterial strain Lactobacillus paracasei DG® (CNCM I-1572) showed enhanced activities compared to the strain Lactobacillus rhamnosus GG (ATCC 53103), that is to say with respect to the probiotic more widely studied and used in literature and documented to exert immunomodulatory properties.

Although the mechanism that supports the Lactobacillus paracasei DG® (CNCM I-1572) antiviral activity observed in this study is unknown, it has been assumed that the rhamnose-rich hetero-exopolysaccharide (EPS) molecule that covers the cells of this bacterium can contribute towards the peculiar cross-talk of Lactobacillus paracasei DG® with the host cells.

Furthermore, it was observed that the combination of the bacterial strains Lactobacillus paracasei DG® (CNCM I-1572) e Lactobacillus paracasei LPC-S01 (DSM 26760) positively modulated the antiviral immune responses to a greater extent with respect to the strain Lactobacillus rhamnosus GG (ATCC 53103), further showing an action in decreasing viral replication and in modulating proinflammatory responses induced by the SARS-CoV-2 virus, even in this case to a greater extent with respect to the strain Lactobacillus rhamnosus GG (ATCC 53103).

1. A composition for use in a method of treating a viral infection of the respiratory system caused by a virus belonging to the species severe acute respiratory syndrome coronavirus (SARS-CoV) in a subject in need, such as a severe acute respiratory syndrome coronavirus and of diseases or symptoms related thereto, wherein said composition comprises:
(i) a mixture (M) comprising or, alternatively, consisting of at least one bacterial strain belonging to the species Lactobacillus paracasei; and, optionally,
(ii) at least one acceptable pharmaceutical grade additive and/or excipient.

2. The composition for use according to claim 1, wherein said at least one bacterial strain belonging to the species Lactobacillus paracasei is selected from the group comprising or, alternatively, consisting of:
a bacterial strain belonging to the species Lactobacillus paracasei identified as Lactobacillus paracasei DG® and deposited at the National Collection of Cultures of Microorganisms of the Pasteur Institute in Paris under the accession number CNCM 1-1572,
a bacterial strain belonging to the species Lactobacillus paracasei identified as Lactobacillus paracasei LPC-S01 and deposited at Deutsche Sammlung von Mikroorganismen und Zellkulturen GmbH (DSMZ) under the accession number DSM 26760, and a mixture thereof.

3. The composition for use according to claim 1 wherein said coronavirus is a virus of the strain severe acute respiratory syndrome coronavirus-2 (SARS-CoV-2) responsible for the disease COVID-2019.

4. The composition for use according to claim 1 , wherein said composition is for oral use.

5. The composition for use according to claim 1 , wherein said mixture (M) consists of Lactobacillus paracasei DG® CNCM 1-1572 strain.

6. The composition for use according to claim 1 , wherein said mixture (M) consists of a Lactobacillus paracasei DG® CNCM 1-1572 strain and a Lactobacillus paracasei LPC-S01 DSM 26760 strain.

7. The composition for use according to claim 1 wherein said mixture M further comprises at least one further bacterial strain selected from the group comprising or, alternatively, consisting of:
a bacterial strain belonging to the species Bifidobacterium breve identified as Bifidobacterium breve BbIBS01 and deposited at Deutsche Sammlung von Mikroorganismen und Zellkulturen GmbH (DSMZ) under deposit number DSM 33231,
a bacterial strain belonging to the species Bifidobacterium breve identified as Bifidobacterium breve BbIBS02 and deposited at Deutsche Sammlung von Mikroorganismen und Zellkulturen GmbH (DSMZ) under deposit number DSM 33232,
a bacterial strain belonging to the species Bifidobacterium animalis identified as Bifidobacterium animalis subsp. lactis B1IBS01 and deposited at Deutsche Sammlung von Mikroorganismen und Zellkulturen GmbH (DSMZ) under deposit number DSM 33233,
a bacterial strain belonging to the species Lactobacillus plantarum identified as Lactobacillus plantarum LpIBS01 and deposited at Deutsche Sammlung von Mikroorganismen und Zellkulturen GmbH (DSMZ) under deposit number DSM 33234,
a bacterial strain belonging to the species Bifidobacterium bifidum identified as Bifidobacterium bifidum MIMBb23sg = BbfIBS01 and deposited at Deutsche Sammlung von Mikroorganismen und Zellkulturen GmbH (DSMZ) under deposit number DSM 32708, and
a mixture thereof.

8. The composition for use according to claim 1 wherein said mixture (M) comprises or, alternatively, consists of a Lactobacillus paracasei DG® CNCM 1-1572 strain and furthermore a further bacterial strain selected from the group comprising or, alternatively, consisting of: Bifidobacterium breve BbIBS01 DSM 33231, Bifidobacterium breve BbIBS02 DSM 33232, Bifidobacterium animalis subsp. lactis B1IBS01 DSM 33233, Lactobacillus plantarum LpIBS01 DSM 33234, Bifidobacterium bifidum MIMBb23sg (= BbfIBS01) DSM 32708 and a mixture thereof.

9. The composition for use according to claim 1 wherein said at least one bacterial strain is a probiotic viable bacterial strain, a paraprobiotic or a postbiotic.

10. The composition for use according to claim 1 wherein said composition further comprises at least one prebiotic; preferably said at least one prebiotic is selected from: inulin, fructooligosaccharide (FOS), galacto-oligosaccharide (GOS), guar gum and mixtures thereof, preferably inulin.

11-12. (canceled)

13. A method of treating a subject for a viral infection of the respiratory system caused by a virus belonging to the species severe acute respiratory syndrome coronavirus (SARS-CoV) in a subject in need, such as a severe acute respiratory syndrome coronavirus and of diseases or symptoms related thereto, comprising administering to the subject a composition comprising:
(i) a mixture (M) comprising or, alternatively, consisting of at least one bacterial strain belonging to the species Lactobacillus paracasei; and, optionally,
(ii) at least one acceptable pharmaceutical grade additive and/or excipient.

14. The method of claim 13, wherein said at least one bacterial strain belonging to the species Lactobacillus paracasei is selected from the group comprising or, alternatively, consisting of:
a bacterial strain belonging to the species Lactobacillus paracasei identified as Lactobacillus paracasei DG® and deposited at the National Collection of Cultures of Microorganisms of the Pasteur Institute in Paris under the accession number CNCM 1-1572,
a bacterial strain belonging to the species Lactobacillus paracasei identified as Lactobacillus paracasei LPC-S01 and deposited at Deutsche Sammlung von Mikroorganismen und Zellkulturen GmbH (DSMZ) under the accession number DSM 26760,
and a mixture thereof.

15. The method of claim 13, wherein said coronavirus is a virus of the strain severe acute respiratory syndrome coronavirus-2 (SARS-CoV-2) responsible for the disease COVID-2019.

16. The method of claim 13, wherein administering is done orally.

17. The method of claim 13, wherein said mixture (M) consists of Lactobacillus paracasei DG® CNCM I-1572 strain.

18. The method of claim 13, wherein said mixture (M) consists of a Lactobacillus paracasei DG® CNCM 1-1572 strain and a Lactobacillus paracasei LPC-S01 DSM 26760 strain.

19. The method of claim 13, wherein said mixture M further comprises at least one further bacterial strain selected from the group comprising or, alternatively, consisting of:
a bacterial strain belonging to the species Bifidobacterium breve identified as Bifidobacterium breve BbIBS01 and deposited at Deutsche Sammlung von Mikroorganismen und Zellkulturen GmbH (DSMZ) under deposit number DSM 33231,
a bacterial strain belonging to the species Bifidobacterium breve identified as Bifidobacterium breve BbIBS02 and deposited at Deutsche Sammlung von Mikroorganismen und Zellkulturen GmbH (DSMZ) under deposit number DSM 33232,
a bacterial strain belonging to the species Bifidobacterium animalis identified as Bifidobacterium animalis subsp. lactis B1IBS01 and deposited at Deutsche Sammlung von Mikroorganismen und Zellkulturen GmbH (DSMZ) under deposit number DSM 33233,
a bacterial strain belonging to the species Lactobacillus plantarum identified as Lactobacillus plantarum LpIBS01 and deposited at Deutsche Sammlung von Mikroorganismen und Zellkulturen GmbH (DSMZ) under deposit number DSM 33234,
a bacterial strain belonging to the species Bifidobacterium bifidum identified as Bifidobacterium bifidum MIMBb23sg = BbfIBS01 and deposited at Deutsche Sammlung von Mikroorganismen und Zellkulturen GmbH (DSMZ) under deposit number DSM 32708, and
a mixture thereof.

20. The method of claim 13, wherein said mixture (M) comprises or, alternatively, consists of a Lactobacillus paracasei DG® CNCM 1-1572 strain and furthermore a further bacterial strain selected from the group comprising or, alternatively, consisting of: Bifidobacterium breve BbIBS01 DSM 33231, Bifidobacterium breve BbIBS02 DSM 33232, Bifidobacterium animalis subsp. lactis B1IBS01 DSM 33233, Lactobacillus plantarum LpIBS01 DSM 33234, Bifidobacterium bifidum MIMBb23sg (= BbfIBS01) DSM 32708 and a mixture thereof.

21. The method of claim 13, wherein said at least one bacterial strain is a probiotic viable bacterial strain, a paraprobiotic or a postbiotic.

22. The method of claim 13, wherein said composition further comprises at least one prebiotic; preferably said at least one prebiotic is selected from: inulin, fructooligosaccharide (FOS), galacto-oligosaccharide (GOS), guar gum and mixtures thereof, preferably inulin.
